(19) United States
(12) Patent Application Publication
(10) Pub. No.: US 2024/0425444 A1
(43) Pub. Date: Dec. 26, 2024

(54) PROCESS FOR PRODUCING CRYSTALLINE L-CARNITINE

(71) Applicants: Hubei Xujie Pharmaceutical Co. Ltd., Jingmen City (CN); Carnitech LLC, North Brunswick, NJ (US)

(72) Inventors: Songzhou Hu, Princeton, NJ (US); Lijun Deng, Hubei (CN); Wen Yu, Hubei (CN); Zhen Song, Shanghai (CN)

(21) Appl. No.: 18/213,482

(22) Filed: Jun. 23, 2023

(51) Int. Cl. C07C 227/42; C07C 229/22

(52) U.S. Cl. CPC C07C 227/42; C07C 229/22; C07B 2200/13

(57) ABSTRACT

There is disclosed a process for the production of crystalline L-carnitine, comprising the step of crystallizing L-carnitine in a mixed solvent comprised of cyclohexane and an alcohol, wherein the alcohol is selected from the group consisting n-propanol, isobutanol, butanol, and a mixture thereof.

### TECHNICAL FIELD

The present invention relates to a process for the production of crystalline L-carnitine. In particular, it relates to a process for the isolation and crystallization of L-carnitine from an aqueous solution.

### BACKGROUND OF THE INVENTION

L-carnitine is a naturally occurring quaternary ammonium acid involved in metabolism in most mammals, plants, and some bacteria. L-carnitine is also called vitamin B_{T} and has a molecular structure of formula (I). It plays a critical role in energy production by transporting long-chain fatty acids into mitochondria so they can be oxidized to produce energy. L-carnitine also plays an important role in the regulation of metabolic pathways involved in skeletal muscle protein balance. Furthermore, L-carnitine acts as an anti-oxidant and as an anti-inflammatory compound. Therefore, L-carnitine finds wide applications as a nutritional supplement and feed additive.

There are many methods for the synthesis of L-carnitine, but only two processes are being used commercially.

The first process is the fermentative oxidation of gamma-butyrobetaine as depicted in the following reaction:

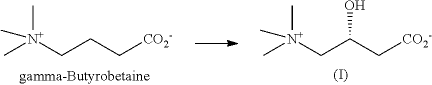

The second process starts from epichlorohydrin and accounts for the majority of L-carnitine produced. The reactions in the second process are described in the following scheme:

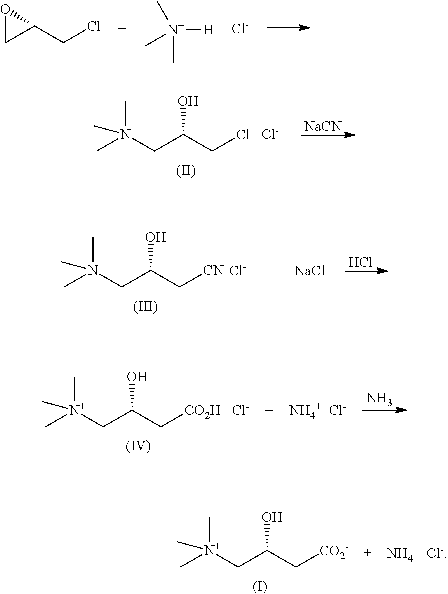

Racemic epichlorohydrin can be efficiently resolved into(S)-epichlorohydrin by using a Jacobsen Co(Salen) catalyst in high yield and high optical purity. (S)-epichlorohydrin is then reacted with trimethylamine hydrochloride to form L-3-chloro-2-hydroxypropyl trimethylammonium chloride (II), which is subsequently reacted with sodium cyanide to form L-carnitinenitrile chloride (III). These two steps can be carried out in a one-pot process without isolating the intermediate (II) in water. After isolation and purification, L-carnitinenitrile chloride is hydrolyzed in concentrated hydrochloric acid to yield L-carnitine and ammonium chloride. L-carnitine is finally isolated from this solution comprised of L-carnitine, excess hydrochloric acid, and ammonium chloride. The process according to this reaction scheme has been described in U.S. Pat. No. 9,096,493.

In the industrial production of L-carnitine, an aqueous solution of L-carnitine is invariably obtained after desalting and further purification. Because L-carnitine is extremely hygroscopic and highly soluble in water, L-carnitine is isolated from this aqueous solution by spray-drying or crystallization from a substantially anhydrous organic solvent.

When spray-drying is used to isolate L-carnitine from an aqueous solution, the product is a fine powder and has a characteristic smell of trimethylamine. In addition, this product can be also slightly colored as spray-drying is performed as a temperature of higher than 120° C. As a result, the product isolated by spray-drying is not suitable for pharmaceutical use. In order to obtain a high purity product, L-carnitine produced by spray-drying may be further purified by recrystallization from an organic solvent.

EP 2325164, U.S. Pat. No. 8,604,237, and WO 2011060903 disclose a process for the purification of L-carnitine by using a mixed solvent. First, L-carnitine is dissolved in a first solvent, in which L-carnitine has high solubility. Suitable first solvents are methanol or ethanol. The solution of L-carnitine in this solvent should be essentially free of water. Preferably, the water content in the solution is below 2%, preferably below 1%, and most preferably 0.5% (w/w). After the solution is seeded with crystals of pure L-carnitine, a second solvent is added, in which L-carnitine is insoluble or has low solubility. Suitable second solvents are acetone, isopropanol, isobutanol, 2-butanone, pentanol, methyl acetate, ethyl acetate, butyl acetate, tetrahydrofuran, toluene, or a mixture thereof. Preferably, the second solvent is acetone. However, this process of using two solvents cannot be used to isolate L-carnitine from an aqueous solution.

WO 2006028068 discloses a process for the purification of L-carnitine by first dissolving L-carnitine in a lower alcohol and water, followed by concentrating under reduced pressure to recrystallize L-carnitine of improved crystalline size and reduced hygroscopicity or by adding another solvent other than the lower alcohol such as ethyl acetate to recrystallize L-carnitine. The amount of water in the mixed solvent can be used to control the particle size of L-carnitine crystal.

EP 0169164 discloses a method for the isolation of L-carnitine from an aqueous solution by concentrating the aqueous solution to dryness in vacuo. The residue is then recrystallized in an ethanol-acetone (2:3) solution to obtain a pure crystalline product of L-carnitine.

U.S. Pat. No. 6,342,034 discloses a process for obtaining a crystalline L-carnitine product from an aqueous solution by removing the water under pressure. The distillation residual was then dissolved in isopropanol at 70° C. and filtered to remove insoluble matter. The resultant material was concentrated again under reduced pressure and recrystallized from a mixed solvent of isopropanol-acetone to obtain the pure form of L-carnitine.

In general, these methods of obtaining L-carnitine are processes in which water is completely distilled off from an aqueous solution of L-carnitine, and then the dried solid is dissolved in an alcohol such as methanol or ethanol and the dissolved L-carnitine is precipitated from a mixed solvent by adding a second solvent such as acetone or ethyl acetate. These methods are not only complicated in operation, but the mixed solvents are difficult to reclaim for reuse.

U.S. Pat. No. 4,708,936 relates to a process for the purification of L-carnitine by using isobutanol. Specifically, an aqueous solution of L-carnitine was concentrated and azeotropically dried by using isobutanol under reduced pressure. The product was then dissolved in isobutanol and partially recrystallized and finally precipitated by adding acetone.

JP2009102258A discloses a method for the purification of L-carnitine from a mixture of L-carnitine and other related components by using a solvent containing butanol for recrystallization.

JP2009102263A relates to a method for the isolation and purification of L-carnitine from an aqueous solution of L-carnitine. Water in an aqueous solution of L-carnitine is replaced with butanol and L-carnitine is crystallized and recrystallized from the same n-butanol. When butanol and an aqueous solution of L-carnitine are mixed, water can be azeotropically removed to below 1% to crystallize L-carnitine. After the separation of L-carnitine, the mother liquor solution is recycled to mix with an aqueous solution of L-carnitine to increase the efficiency and recovery yield of L-carnitine. However, if the temperature to replace water with n-butanol is above 80° C., the yield of L-carnitine is decreased. As a result, the temperature for the entire process has to be maintained below 80° C. Hence, the distillation of water and butanol must be carried out under reduced pressure. However, azeotropic distillation under reduced pressure is complicated in operation and the loss of solvent becomes significant.

It is an object of the present invention to ameliorate these disadvantages and to disclose an improved process for the production of L-carnitine from an aqueous solution. It is another object of the present invention to disclose a process for the purification of L-carnitine.

### SUMMARY OF THE INVENTION

The present invention discloses a process for the production of L-carnitine from an aqueous solution. The present invention further relates to a process for the purification of L-carnitine. The invention is accomplished by crystallizing L-carnitine in a mixed solvent comprised of cyclohexane and an alcohol, wherein the alcohol is selected from the group consisting of n-propanol, isobutanol, n-butanol, and a mixture thereof. Water in the solution can be efficiently and economically removed by azeotropic distillation to crystallize L-carnitine.

### DETAILED DESCRIPTION OF THE INVENTION

The present invention relates to a process for the production of L-carnitine. In particular, it relates to a process for the production of crystalline L-carnitine from an aqueous solution of L-carnitine by crystallization and recrystallization in a specific solvent, in which the removal of water is performed under atmospheric pressure and at a temperature below the decomposition temperature of L-carnitine.

After extensive investigation, it has been found that crystalline L-carnitine can be isolated from an aqueous solution in a mixed solvent that is comprised of cyclohexane and an alcohol, wherein the alcohol is selected from the group consisting of n-propanol, isobutanol, butanol, and a mixture therefore. It is surprising and unexpected to discover that n-propanol, isobutanol, butanol, and their mixture are suitable for the azeotropic removal of water from an aqueous solution of L-carnitine, but ethanol and isopropanol are not suitable.

Preferably, the alcohol is n-propanol. When n-propanol is used as the alcohol, azeotropic distillation temperature does not exceed 80° C. and water in the solution can be effectively removed. Moreover, n-propanol provides an added advantage in that n-propanol has a pleasant odor profile that is similar to ethanol.

The process according to the present invention comprises the steps of: (1) preparing an aqueous solution of L-carnitine; (2) mixing the aqueous solution with the alcohol and cyclohexane; (3) azeotropically distilling the solution to remove water from the solution; (4) cooling the solution or crystalline suspension to crystallize L-carnitine; and (5) isolating the crystalline L-carnitine by a solid-liquid separation to yield a mother liquor solution.

L-carnitine used in the production and purification in the process according to the present invention is not particularly limited. Suitable aqueous solutions of L-carnitine can be obtained by any well-known process, for example, a hydrolysis solution of L-carnitinenitrile chloride, a solution from the optical resolution of carnitine, a solution from the optical resolution of carnitinenitrile chloride or carnitinamide chloride, a L-carnitine solution obtained by asymmetric hydrogenation of suitable precursors, a L-carnitine solution from fermentation, and a solution of L-carnitine subjected to ion exchange resin treatment or electrodialysis.

The concentration of L-carnitine in an aqueous solution is not limited. The concentration of L-carnitine can be adjusted by concentrating a dilute aqueous solution by performing an operation of concentration. Preferably, the concentration of L-carnitine is from 30% to 90% (wt/wt). More preferably, the concentration of L-carnitine is from 50% to 90%.

The aqueous solution of L-carnitine is mixed with an alcohol selected from the group consisting of n-propanol, isobutanol, butanol, and a mixture thereof, followed by cyclohexane. On the other hand, a mixed solution of the alcohol and cyclohexane can be added to an aqueous solution of L-carnitine.

The amount of an alcohol relative to the amount of L-carnitine is not limited. The concentration of L-carnitine in the solution of an alcohol is from 10% to 90% (wt/wt). Preferably, the concentration is from 20% to 80%, more preferably from 30% to 60%, most preferably from 30% to 50%.

The amount of cyclohexane relative to the amount of an alcohol is not limited. Preferably, cyclohexane is used in a concentration from 5% to 60% (wt/wt) of the alcohol.

In order to crystallize L-carnitine, it is necessary to reduce the content of water to a concentration below 5% (wt/wt), preferably below 3%, more preferably below 2%, most preferably below 1%. In the process according to the present invention, this objective can be achieved by azeotropic distillation at atmospheric pressure. It has been surprising and unexpected to discover that the presence of L-carnitine in the solution renders the azeotropic distillation efficient and successful. It has been found that the boiling temperature of the solution does not exceed 80° C. and the distillate is easily separated into an upper phase that contains not more than 2% of water and a lower phase that is mainly water, if n-propanol is used as the alcohol. During the azeotropic distillation, the upper phase can be continuously returned to the L-carnitine solution until the water content is reduced to a desired level, preferably below 2%, more preferably below 1%.

During the azeotropic distillation, crystalline L-carnitine may be formed. After the azeotropic distillation, the crystalline suspension is then cooled to complete the crystallization of L-carnitine. On the other hand, the hot solution may be cooled to crystallize L-carnitine.

Crystalline L-carnitine can be isolated by means of a solid-liquid separation technique, such as filtration, centrifugation, or press filtration. The solid L-carnitine can be recrystallized or dried to yield the product.

After the separation of crystalline L-carnitine, a mother liquor solution is obtained. To the mother liquor solution can be added additional cyclohexane to precipitate additional L-carnitine. On the other hand, this mother liquor solution may be concentrated and cooled to crystallize additional L-carnitine. Preferably, this mother liquor solution is mixed with another batch of an aqueous solution of L-carnitine. The solution is then subject to azeotropic distillation to remove water to achieve the crystallization of L-carnitine. This cyclic process according to the present invention can recover L-carnitine from an aqueous solution in a high yield, preferably greater than 90%, more preferably greater than 95%, most preferably nearly quantitative 100%.

It is important to note a distinct advantage of the process according to the present invention that the mother liquor solution can be cyclically used without separating each individual component in the mixed solvent.

The process according to the present invention can also be used to purify solid impure L-carnitine to yield a pure crystalline product. For example, when solid L-carnitine is obtained from spray-drying of an aqueous solution of L-carnitine, the product appears slightly discolored and smells of amine. This form of L-carnitine can be readily purified by recrystallizing from a mixed solvent of cyclohexane and an alcohol, wherein the alcohol is selected from the group consisting of n-propanol, isobutanol, butanol, and a mixture thereof.

The recrystallization of L-carnitine in the mixed solvent in the process according to the present invention is particularly advantageous. First, the water content in the solution of L-carnitine in the mixed solvent can be readily controlled by an azeotropic distillation. Second, the mother liquor solution after the separation of crystalline L-carnitine can be cyclically used without separating each component, thus greatly simplifying the process. Third, impure L-carnitine can be readily purified to pure L-carnitine.

After L-carnitine is isolated, the L-carnitine can be converted to L-carnitine L-tartrate by reacting with L-tartaric acid, L-carnitine fumarate with fumaric acid, acetyl-L-carnitine hydrochloride with acetyl chloride, and propionyl-L-carnitine hydrochloride with propionyl chloride.

The process according to the present invention can be carried out discontinuously, semi-continuously, or continuously.

### EXAMPLES

The following examples will illustrate the practice of this invention but are not intended to limit its scope.

### Comparative Example 1

To a round-bottom flask equipped with a Dean-Stark distillation head were added 66 g of 75% L-carnitine solution, 200 mL of ethanol, and 50 mL of cyclohexane. After the solution was refluxed at internal temperature of 66° C. for 8 hours, the water content in the flask reached 6.0%. L-carnitine did not crystallize from the solution.

### Comparative Example 2

To a round-bottom flask equipped with a Dean-Stark distillation head were added 66 g of 75% L-carnitine solution, 200 mL of isopropanol, and 50 mL of cyclohexane. After the solution was refluxed at internal temperature of 75° C. for 8 hours, the water content in the flask reached 6.8%. L-carnitine did not crystallize from the solution.

### Example 1

To a round-bottom flask equipped with a Dean-Stark distillation head were added 214 g of 75% L-carnitine solution, 860 mL of n-propanol, and 214 mL of cyclohexane. After the solution was azeotropically refluxed at internal temperature of 79° C. for 8 hours, the water content in the flask reached 1.0%. The solution was slowly cooed to 20° C. to crystallize L-carnitine. The crystalline suspension was filtered and dried to obtain 72.4 g of a white crystalline L-carnitine solid after drying in a yield of 45.3%. [α]_{D}²⁵=−31.6° (c=10, H₂O), water content: 0.21%. The mother liquor solution weighted 810 g.

### Example 2

To a round-bottom flask equipped with a Dean-Stark distillation head were added the mother liquor solution of Example 1, 200 g of 75% L-carnitine solution, 50 mL of n-propanol, and 13 mL of cyclohexane. After the solution was azeotropically refluxed at internal temperature of 79° C. for 8 hours, the water content in the flask reached 1.2%. The solution was slowly cooed to 20° C. to crystallize L-carnitine. The crystalline suspension was filtered and dried to obtain 143 g of a white crystalline L-carnitine solid after drying in a yield of 95.3%. [α]_{D}²⁵=−31.3° (c=10, H₂O); water content: 0.25%. The mother liquor solution weighted 804 g.

### Example 3

To a round-bottom flask equipped with a Dean-Stark distillation head were added the mother liquor solution of Example 2, 200 g of 75% L-carnitine solution, 50 mL of n-propanol, and 13 mL of cyclohexane. After the solution was azeotropically refluxed at an internal temperature of 79° C. for 8 hours, the water content in the flask reached 0.9%. The solution was slowly cooed to 20° C. to crystallize L-carnitine. The crystalline suspension was filtered and dried to obtain 146.3 g of a white crystalline L-carnitine solid after drying in a yield of 97.5%. [α]_{D}²⁵=−30.8° (c=10, H₂O); water content: 0.27%. The mother liquor solution weighted 801 g.

### Example 4

To a round-bottom flask equipped with a Dean-Stark distillation head were added 66 g of 75% L-carnitine solution, 200 mL of isobutanol, and 50 mL of cyclohexane. After the solution was refluxed at an internal temperature of 90° C. for 8 hours, the water content in the flask reached 1.3%. The solution was slowly cooed to 20° C. to crystallize L-carnitine. The crystalline suspension was filtered and dried to obtain 42.5 g of a white crystalline L-carnitine solid after drying. [α]_{D}²⁵=−30.5° (c=10, H₂O); water content: 0.23%.

### Example 5

To a round-bottom flask equipped with a Dean-Stark distillation head were added 66 g of 75% L-carnitine solution, 200 mL of n-butanol, and 50 mL of cyclohexane. After the solution was refluxed at an internal temperature of 95° C. for 8 hours, the water content in the flask reached 1.2%. The solution was slowly cooed to 20° C. to crystallize L-carnitine. The crystalline suspension was filtered and dried to obtain 40.7 g of a white crystalline L-carnitine solid after drying. [α]_{D}²⁵=−30.9° (c=10, H₂O); water content: 0.18%.

It will be understood that the foregoing examples and explanation are for illustrative purposes only and that various modifications of the present invention will be self-evident to those skilled in the art. Such modifications are to be included within the spirit and purview of this application and the scope of the appended claims.

What is claimed is:

1. A process for the production of crystalline L-carnitine comprising the step of crystallizing L-carnitine in a mixed solvent comprised of cyclohexane and an alcohol; wherein the alcohol is selected from the group consisting of n-propanol, isobutanol, butanol, and a mixture thereof.

2. The process according to claim 1, wherein the L-carnitine in an aqueous solution is mixed with the mixed solvent and azeotropically distilled to remove water.

3. The process according to claim 1, wherein the alcohol is n-propanol.

4. The process according to claim 1, wherein the alcohol is isobutanol.

5. The process according to claim 1, wherein the alcohol is n-butanol.

6. The process according to claim 1, wherein the solution of L-carnitine in the mixed solvent contains less than 5% of water by weight.

7. The process according to claim 1, wherein the solution of L-carnitine in the mixed solvent contains less than 3% of water by weight.

8. The process according to claim 1, wherein the solution of L-carnitine in the mixed solvent contains less than 1% of water by weight.

9. The process according to claim 1, wherein the L-carnitine contains cyclohexane as a residual solvent.

10. The process according to claim 1, wherein the L-carnitine is free of ethanol, acetone, and isopropanol.

11. The process according to claim 3, wherein the L-carnitine contains n-propanol as a residual solvent.

12. The process according to claim 4, wherein the L-carnitine contains isobutanol as a residual solvent.

13. The process according to claim 5, wherein the L-carnitine contains n-butanol as a residual solvent.

14. The process according to claim 1, further comprising the production of L-carnitine L-tartrate by reacting the L-carnitine with L-tartaric acid.

15. The process according to claim 1, further comprising the production of L-carnitine fumarate by reacting the L-carnitine with fumaric acid.

16. The process according to claim 1, further comprising the production of acetyl-L-carnitine hydrochloride by reacting the L-carnitine with acetyl chloride.

17. The process according to claim 1, further comprising the production of propionyl-L-carnitine hydrochloride by reacting the L-carnitine with propionyl chloride.
